(19) United States
(12) Patent Application Publication
(10) Pub. No.: US 2021/0335456 A1
(43) Pub. Date: Oct. 28, 2021

(54) SYSTEMS AND METHODS FOR COLLECTING CLINICAL AND NON-CLINICAL DATA ACROSS MULTIPLE FUNCTIONAL MODULES THROUGH A CENTRAL GATEWAY

(71) Applicants: Avik Kumar Pal, Fremont, CA (US); Yerramalli Subramaniam, Pleasanton, CA (US)

(72) Inventors: Avik Kumar Pal, Fremont, CA (US); Yerramalli Subramaniam, Pleasanton, CA (US)

(73) Assignee: CliniOps, Inc., Fremont, CA (US)

(21) Appl. No.: 16/857,181

(22) Filed: Apr. 24, 2020

(51) Int. Cl. G16H 10/20; G16H 10/60; G06F 16/907

(52) U.S. Cl. CPC G16H 10/20; G06F 16/907; G16H 10/60

(57) ABSTRACT

Systems and methods for collecting clinical trial data across multiple functional modules is described. One described method comprises managing clinical and non-clinical data on a client device over a network; the data is linked through a central gateway across a plurality of functional modules; with access control to each functional stake holder; and transmitting the data, for access to sponsors over the network.

### FIELD OF THE INVENTION

The present invention relates generally to clinical trial data collection. This invention more particularly relates to the management of the clinical trial data across multiple clinical trial functional modules through a central gateway.

### BACKGROUND

Clinical trials is one of the most critical element of the drug development process, where the average drug-to-market is usually more than a decade and costs can sometimes go upwards of 2 billion US dollars to bring a new drug to market. Clinical trials account for 60-70% of the time and costs incurred in drug development, which is a major driver of the high cost and long duration of bringing new drugs to market.

Conducting a successful clinical trial is a very complex process due to the collaborative nature, multiple stakeholders, multi variate data sources and the high stakes involved in running a clinical trial.

Clinical and non-clinical data is collected for different modules to successfully conduct a clinical trial. Over the years, various modules have been developed to address the requirements for different functionalities namely EDC (electronic data collection), ePRO (electronic patient reported outcome), eICF (electronic informed consent form), CTMS (clinical trial management system), IRT (interactive response technology). All these modules have historically been developed in silos to address the specific need of the specific stakeholder or user role. But each of these modules cannot work in silos, and hence a lot of complex system integrations, system validation and data exchanges happen, adding a whole lot of complexity to the already complex clinical trial process.

A wide range of coordination is also needed that involves the sites (hospitals or health centers), the subjects (patients or participants), the principal investigators (physicians or researchers), the site coordinators (nurses or care givers), the sponsors (the pharma, biotech or medical device company that is sponsoring the clinical trial), the clinical research organizations (CRO) who are providing the services to run these large multi-country global clinical trial, the biostatisticians who analyze the data collected during the trial and the regulatory bodies (FDA, EMA) who review the final data submitted for a go/no-go decision or approval of the drug to market.

There is a need to rethink on how to design an efficient systems and methods, across multiple functional modules, data requirements and stakeholder roles and responsibilities in mind.

### SUMMARY

Embodiments of the present invention provide systems and methods for collecting clinical trial data across multiple functional modules. In one embodiment of the present invention, a method comprises managing clinical and non-clinical data on a client device over a network; the data is linked through a central gateway across a plurality of functional modules; with access control to each functional stake holder; and transmitting the data, for access to sponsors over the network.

This embodiment is illustrative and is an example to help with the understanding thereof. Illustrative embodiments are discussed in the Detailed Description section.

### FIGURES

The benefits, needs and advantages of this current invention is better understood, when the following Detailed Description below is read with reference to the drawings in this section, wherein:

FIG. 1 is a schematic diagram illustrating the functional architecture, in one embodiment of the present invention, to collect data across multiple modules through a central gateway

FIG. 2 is a block diagram illustrating the system architecture, in one embodiment of the present invention, to collect data across multiple modules through a central gateway

FIG. 3 is a schematic representation of the current challenges of siloed design, with difficult to manage interdependency between functional modules, to conduct a specific clinical trial

FIG. 4 is a screenshot of the mobile application, in one embodiment of the present invention, to collect patient data, across different patient visits

FIG. 5 is a screenshot of the case report forms (CRF) data collected by the healthcare team on the mobile application, displayed through the central gateway, on the EDC module of the study portal

FIG. 6 is a screenshot of the data collected by patients themselves on the mobile application, displayed through the central gateway, on the ePRO module of the study portal

FIG. 7 is a screenshot of the patient signatures collected as part of the informed consent process, on the mobile application, displayed through the central gateway, on the eICF module of the study portal

FIG. 8 is a screenshot of the auto-coded terms of the data collected during the study, against standard coding libraries, displayed through the central gateway, on the Coder module of the study portal

FIG. 9 is a screenshot of randomized patients in different treatment arms, that can be displayed through the central gateway, on the IRT module of the study portal

FIG. 10 is a screenshot of the clinical trial management system functionalities and reports, cutting across multiple modules, displayed through the central gateway, on the CTMS module of the study portal

### DETAILED DESCRIPTION

Embodiments of the present invention comprise systems and methods to manage multiple functional modules and functional stakeholder activities via a single central gateway. Over the years, several applications evolved to address different functional areas of the clinical trial processes, each developed in silos, to manage certain activities of the process. Each of these applications are extremely interdependent to support a smooth functioning of the trial conduct. Hence the industry spends a lot of effort in integration, validation and data transfers across applications, which not only adds a lot of cost burden but also makes the process even more complex. The proposed design with the central gateway, brings multiple functional modules on a common platform, and eliminates the need for siloed applications and associated integrations.

Over the decades, regulators and pharmaceutical industry groups have been interested in electronic source (eSource) in clinical trials as an effort towards digitizing clinical trials. eSource may provide efficiencies and value; however, eSource adoption is fragmented and slow. Acceleration of eSource adoption is a critical step in modernizing the conduct of clinical trials. The desired future state is one in which all source data, acquired across multiple systems are electronic, adequate in quality, fully acceptable in clinical trial submissions by regulators, and completely inter-operable, without restrictions.

To realize this vision, one such embodiment provides the ability to have multiple functional modules architected in a way that facilitates seamless data structure encompassing multiple functionalities and stake holder responsibilities in a single database structure, that align upon guidance to promote data integrity, data privacy, data security, and data interoperability. Furthermore, the architecture will improve data integrity significantly by allowing direct data flow from the source to the sponsor's system, with minimal or no human intervention.

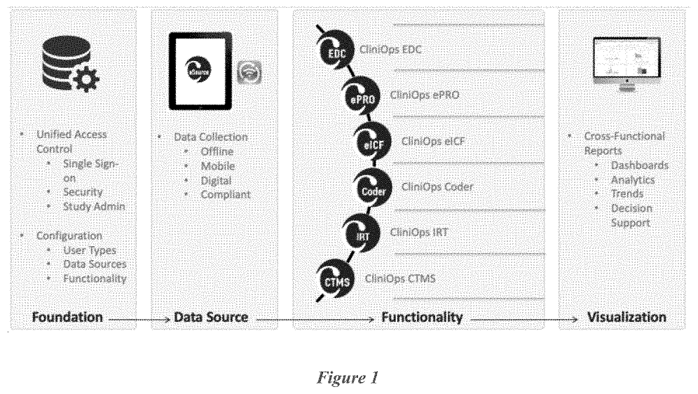

FIG. 1 is a schematic diagram illustrating the functional architecture, to collect clinical and non-clinical data at hospitals on client devices across multiple functional modules, and sent via a server to a central gateway.
The Foundation layer is where all the configurations are done centrally which is then accessed across all the applications. This includes but is not limited to the central access control, security, user administration, data sources, functionality, the case report forms, checks and balances, consent forms et al.
The Data Source layer is also designed to leverage a single point of data collection used across multiple functional modules. This includes data collected in case report forms, data collected from labs, from med devices, from other connected devices, from radiologists, from patient signatures (for informed consent) and any data entry that happens as part of the process.
The Functional layer constitutes one of the current embodiments with multiple different modules that performs the functionality designed to leverage the functional and data sourcing layer. The different functional modules include:
   a client device; and
   a collection of clinical and non-clinical data; and
   wherein the said clinical and said non-clinical data on the said client device is collected at a hospital location; and
   wherein the said clinical and said non-clinical data on the said client device is collected at the said hospital location, across a plurality of functional modules; and
   wherein one of said plurality of functional modules is an electronic data collection (EDC) system; and
   wherein one of said plurality of functional modules is an electronic patient reported outcome (ePRO) system; and
   wherein one of said plurality of functional modules is an electronic informed consent (eICF) system; and
   wherein one of said plurality of functional modules is an automatic medical coding (Coder) system; and
   wherein one of said plurality of functional modules is an interactive response technology (IRT) system; and
   wherein one of said plurality of functional modules is a clinical trial management system (CTMS) system; and
   wherein the said clinical and said non-clinical data on the said client device is collected at the said hospital location, across the said functional modules, with access control to each functional stake holder; and
   wherein linking the said clinical and said non-clinical data, collected at the said hospital location, across the said functional modules, through a central gateway; and
   wherein transmitting the said clinical and said non-clinical data to the server over the network, for access to sponsors.
The clinical and non-clinical data collected across the different functional modules, is then provided access control to each functional stake holder through a central gateway; and transmitted over the network, for access to sponsors.
The visualization layer is the final layer that displays the dashboards graphs, charts and reports across any of the applications as needed. It also has intelligent decision support capabilities to help with advanced reports across multiple modules.

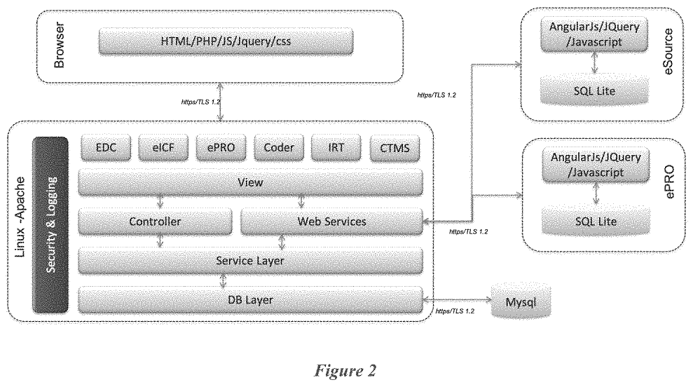

FIG. 2 is a block diagram illustrating the system architecture, to collect data from multiple source systems through a central gateway. It illustrates how multiple types of datasets can be sourced electronically and architected into a central gateway, from where they can be viewed through a single portal. A central access control determines what data set can be viewed by what roles, based on user permissions.

To address the challenges and costs of an end to end clinical trial process, the design of an integrated architecture is an important step. Over the years, industry has witnessed new innovative products and solutions in the form of social media, collaboration platform, security solution, analytics, cloud solution, AI/ML, block chain. The architecture of the said embodiment has been designed keeping in mind the above considerations. The architectural layers that have been used, can broadly be described as follows:

User Access Layer
The user access layer has two points of entry viz the web portal and the mobile interface (MI). The admin, investigators and patients will mostly access the web portal where as the sub-investigators or healthcare professionals will use the mobile application for subject registration and entering visit data. Sub-investigators can also access the web portal for generating reports. LAMP (Linux, Apache, MySQL, PHP) stack has been used for implementing the web portal. The data collection requirement is supported by this layer where subject information for CRF is collected at site locations. The data is collected via mobile devices.

Application Layer
The application layer is responsible for mapping of db entities with domain objects. The web service interacts with the application layer to serve the mobile request whereas the controller interacts with application layer to serve the web portal. Zend framework has been used for the MVC design and component library. The layout layer provides a composite view for the final rendering of the page in web browser. The helper layer mostly contains utility classes where common utility classes and functions are kept.

Security Layer
The current innovation of the product architecture has been designed to address several security features at different levels. This includes the application level security, the database level security and infrastructure level security. The broad features of the security architecture are:
   Encryption of DB data
   Encryption of data in motion conforming to TLS 1.2
   PHI (Protected Health Information) data encrypted at MI. Also, role-based access designed for PHI information.
   Platform to support the capability to enforce password and remote locking for endpoint MI
   Both authentication and authorization are supported at the portal end and MI.
   The entire infrastructure uses firewalls to segregate network zones to protect environment from external threats
   The platform adheres to NIST 800-131 guidelines for secure encryption
   The architecture supports encryption over the wire
   The platform leverages common Certifying Authorities for any user requests operating over secure channels
   MI application is distributed over the air to site user mobile devices using MDM (Mobile device management) solution provider
   The system provides for alert generation and error notification

Database Layer
The backend architecture supports varied operating systems and support for different programming languages. The system is responsive to queries and provide support for large data sets. For example my SQL that has been used by the application supports large databases, up to 50 million rows or more in a table. The default file size limit for a table is 4 GB, but it can be increased (if the operating system can handle it) to a theoretical limit of 8 million terabytes (TB). The DB should be also customizable to allow programmers to modify the backend to suit their needs.

Infrastructure Layer
The infrastructure is designed such as to provide for the necessary abstraction for the entire system components while executing business functions. Training requirements of the various stakeholders have been addressed and the product architecture has been designed to restrict access to training areas based on role permissions, The architecture is designed to include integration, testing/validation and support for development, QA and production environments and each environment is such as to support same functionality on a software level. Also, the production environment includes a disaster recovery environment. Access to hosting environment is controlled by a private key. Hosting environment provides secured remote access to key administrative platforms.
Key Architecture Goals: The key architectural goals that have been addressed in are:
   Service Orientation: To ensure extensible architecture, the design is calable to independently build, assemble, deploy and consume specific business services for varied stakeholder needs. The application supports the same as can be tested against the various study integration of different companies as well as seamless integration of different MI.
   Extensibility: The application architecture has been designed so as to seamlessly integrate with new plug-ins as well as new solutions in the industry. This can be tested by integrating the application with blue tooth enabled devices as well as testing for functionality against the different MI's. New applications can be added as new modules and leverage the existing service and DB layers to achieve ‘single-source-of truth’ via a central gateway.
   Scalability: At all the above described layers the platform provides scalability. This can be thoroughly tested by increasing the number of global users on the system and monitoring the response time of the system to be within a reasonable tolerable limit.
   Security: Message confidentiality as well as message integrity is maintained between two or more end points. System security can tested against the security of each of the product components.
   Availability: The integrated product platform ensures high availability of its resources and is able to handle additional global users.
   Response Time: The response time is reasonable for regular transactions when investigators, sponsors, users perform clinical study planning and document exchange. This can be tested for the entire workflow (both for web and MI) to keep the response time less than a specified time.

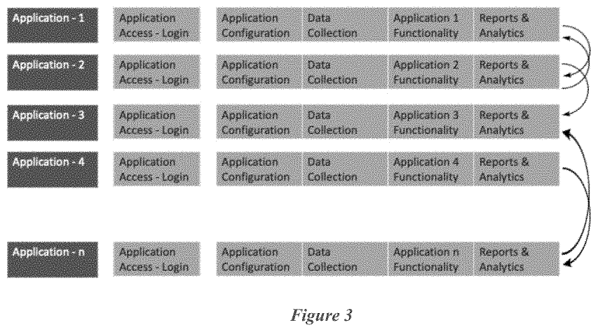

FIG. 3 is a schematic representation of the current state, where Application 1, 2, . . . n represents the different applications (EDC, ePRO, eICF . . . et al), that are currently used to support a clinical trial. Each application is designed to have its own user access, control, its own configuration, data ingestion, functionality and visualization. Unfortunately, most applications are dependent on each other and hence, a lot of integration efforts are needed to feed data from one system to the other. This poses several data challenges resulting in poor data quality, time to port data from one system to the other and to maintain the system integrations with multiple points of failure in such a setup.

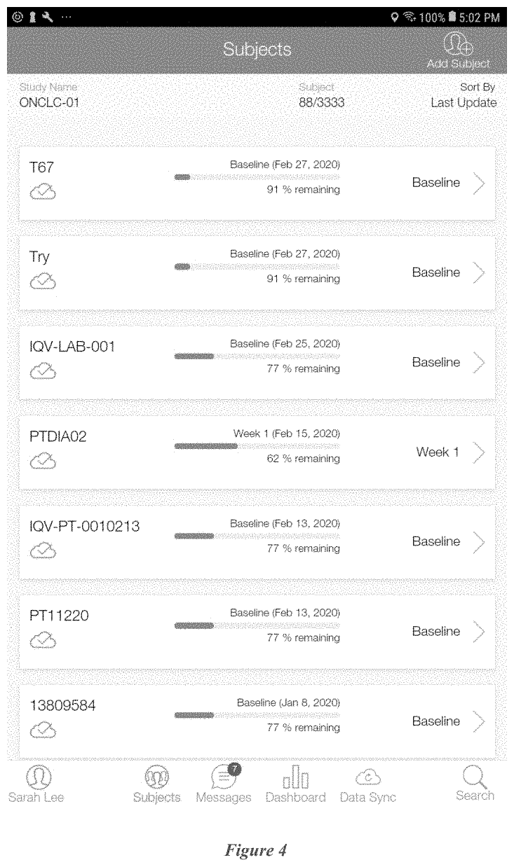

FIG. 4 is a screenshot of the mobile eSource module, in one embodiment of the present invention, to collect patient data, across patient visits. Once the patient ID is selected, all the visits are shown that are associated with that patient including the dates of the visits, known as subject calendar. Each visit then represents the forms that are tied to that visit, and each form has all the data fields that needs to be collected during that visit. To ensure seamless data collection on digital tablets in ambulatory settings where the doctor has to constantly go from bed to bed or patient to patient, the system has been designed to be completely functional without the dependency on internet, which is sometimes a challenge in spotty zones or highly secured networks in hospital settings. Data is then synchronized at regular intervals with the central portal. The data is encrypted following all data security standards both at rest and in motion. FIG. 4 also shows how the hospital is doing in terms of subject enrollment, any pending queries from the sponsor and what data has been synchronized with the central database.

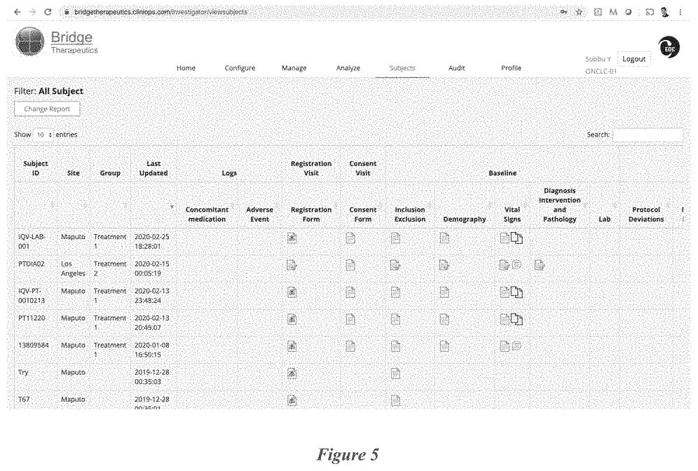

FIG. 5 is a screenshot of the Case Report Forms (CRF) status, as collected by the care team on the mobile devices, displayed on the EDC module of the study portal. Each row represents a unique patient, along with the site, treatment group and the last update date of the patient data. It also shows the status of the different forms that the patient has completed including the form status as depicted by the icons associated with each form. The built-in workflow displays the form status at a glance:
Fully filled
Partially filled
Filled with errors
Reviewed
Locked
PI sign-off

Clicking on the form icon opens the form for review, follow-up action, or next steps as needed. Once the forms are in a locked state, the PI sign-off is initiated. Upon completion of the trial, data can be exported in various formats as appropriate for analysis and long term archival—these may include but not limited to CSV, XML, XPT, PDF and other formats.

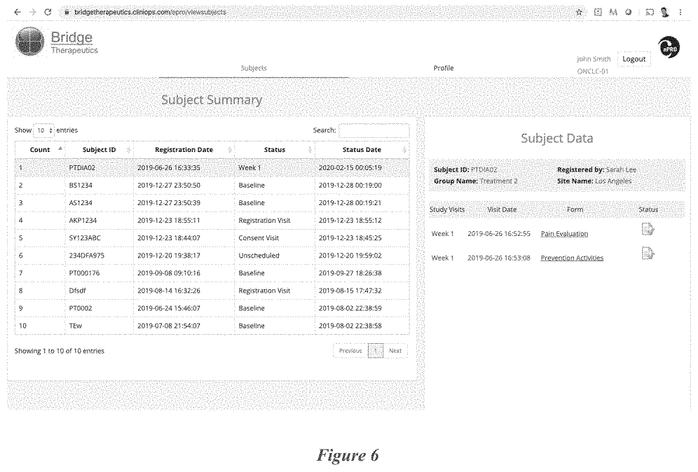

FIG. 6 is a screenshot of the data collected by patients themselves on the smartphone devices, displayed on the ePRO module of the study portal. Access to fill these forms are specifically given to patients themselves. The data collected from these forms can then be aggregated to the ePRO module of the application and available through the central gateway for a wholistic analysis of the patient.

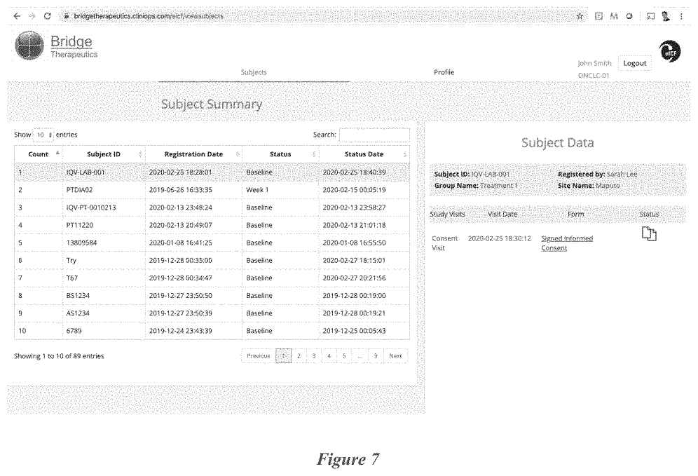

FIG. 7 is a screenshot of the patient signatures collected as part of the informed consent process, on the mobile devices, displayed on the eICF module of the study portal. The informed consent contains PHI (protected health information) data and the system is designed to effectively handle such sensitive information. The patient signatures are collected on the mobile device along with system time stamp and geo-location, the module has other functionalities to ensure that the patient had a good understanding of the risks and benefits of joining such a trial. Once the process is completed, a signed informed consent with all relevant information is automatically generated and a digital/paper copy of the same is handed over to the patient as per ICH guidelines.

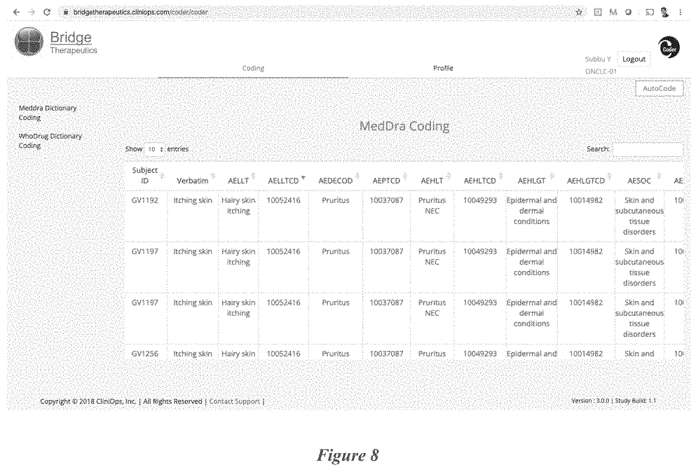

FIG. 8 is a screenshot of the auto-coding functionality. Data collected during the study is automatically coded against standard coding libraries (MedDRA & WHODrug), and the coded terms are displayed on the Coder module of the study portal.

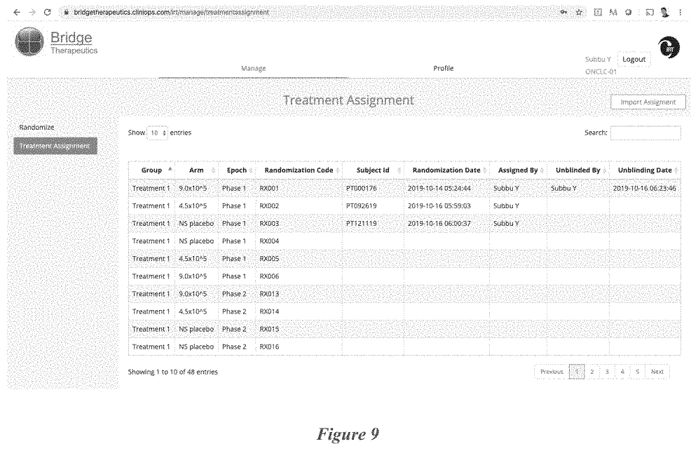

FIG. 9 is a screenshot of randomized patients in different treatment arms, that can be displayed on the interactive response technology (IRT) module of the study portal. This module provides support for both simple and advanced randomization algorithms (permuted blocked stratification, minimization). The functionality provides support for double blinded studies, option for tracking kits, emergency unblinding and several other features. The standardized reports can randomize patients and track their progress in real-time

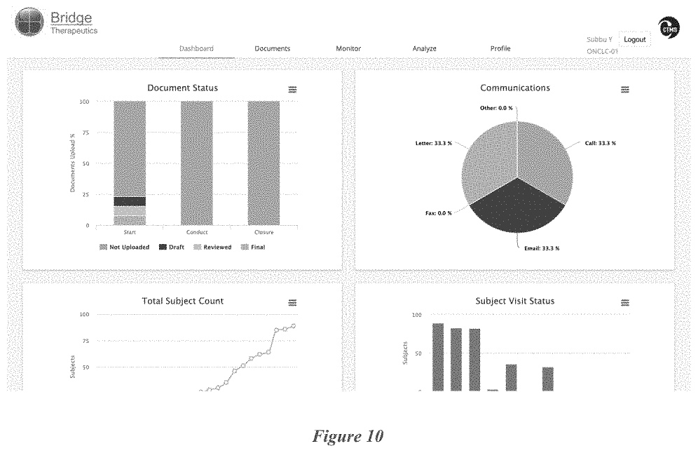

FIG. 10 is a screenshot of the Clinical Trial Management System (CTMS) functionalities and reports, across multiple modules can be displayed on the CTMS module of the study portal. Usually CTMS requires data across all the other systems like EDC, ePRO, eICF etc, and there is a significant effort to feed the CTMS system with such data. But the current architecture with the central gateway enables the CTMS module to fetch data from any of the other modules as needed to generate cross-module reports in real-time.

The application is purpose-built to address several data challenges and the challenges of the data flow across various different applications, currently prevalent in large global clinical trials. It streamlines ‘clinical data management’ with ‘clinical operations’, and accelerates ‘regulatory submission’ functions across the different stakeholders. The efficiency and quality of a lot of the down-stream activities of the clinical trial workflow, depends on high quality & real-time ‘data collection’ at source, which is the first step in the ‘journey of the data’. The embodiments of this invention address this challenge, and as a result, the sponsors and CROs can view the data along with powerful dashboards, reports and study KPIs, via a web portal across all modules and functionalities as part of the system. In addition, there are a host of innovative features that fully automates and streamlines the clinical trial workflow, including downstream activities such as data management, query resolution and statistical programming.

Some embodiments of the present invention are compliant with some or all of both HIPAA and FDA 21 CFR Part 11 regulations. FDA 21 CFR Part 11 covers all aspects of electronic records including signatures, integrity and authenticity, record creation, audit trails and archiving of data. Part 11 requires electronic records that are “created, modified, maintained, archived, retrieved, or transmitted, under any records requirement set forth in agency regulations” may be protected by procedures and controls to “ensure the authenticity, integrity, and, when appropriate, the confidentiality of electronic records, and to ensure that the signer cannot readily repudiate the document as not genuine.” The goal of Part 11 is to ensure electronic records and signatures are authentic and traceable. Without the rule, accidental or deliberate tampering with electronic patient records would be difficult to monitor.

The above description of the embodiments of the invention has been presented for the purpose of illustration and description and is not intended to be exhaustive or to limit the invention to the precise apps disclosed. Modifications and additional features and capabilities thereof may be apparent, without departing from the spirit and scope of the present invention.

That what is claimed:

1. A method comprising:
a client device; and
a collection of clinical and non-clinical data; and
wherein the said clinical and said non-clinical data on the said client device is collected at a hospital location; and
wherein the said clinical and said non-clinical data on the said client device is collected at the said hospital location, across a plurality of functional modules; and
wherein one of said plurality of functional modules is an electronic data collection (EDC) system; and
wherein one of said plurality of functional modules is an electronic patient reported outcome (ePRO) system; and
wherein one of said plurality of functional modules is an electronic informed consent (eICF) system; and
wherein one of said plurality of functional modules is an automatic medical coding (Coder) system; and
wherein one of said plurality of functional modules is an interactive response technology (IRT) system; and
wherein one of said plurality of functional modules is a clinical trial management system (CTMS) system; and
wherein the said clinical and said non-clinical data on the said client device is collected at the said hospital location, across the said functional modules, with access control to each functional stake holder; and
wherein linking the said clinical and said non-clinical data, collected at the said hospital location, across the said functional modules, through a central gateway; and
wherein transmitting the said clinical and said non-clinical data to the server over the network, for access to sponsors.

2. The method of claim 1, wherein the said client device is a processor-based device that can be connected to the said network,
wherein the processor-based device is a computer; and
wherein the processor-based device is a tablet; and
wherein the processor-based device is a smartphone.

3. The method of claim 1, wherein the said clinical and said non-clinical data is transmitted to the said server through the said network through secured protocols, for access to the said sponsors,
wherein the said network may include https with TLS; and
wherein the said network may include websockets using wss; and
wherein the said network may include sftp.

4. The method of claim 1, wherein the said sponsors may include pharmaceutical, biotech, medical devices companies, academic institutions or research organizations running clinical trials.

5. The method of claim 1, wherein the collection of the said clinical and said non-clinical data on the said client device, via integration with medical devices.

6. The method of claim 1, further ensuring the said healthcare professional at the said hospital can seamlessly interact with the said sponsor.

7. A system comprising:
wherein the said client device in communication with the said server device over the said network; and
wherein the said clinical and said non-clinical data captured at the said hospital is transmitted over the said server; and
wherein the said clinical and said non-clinical data is transmitted over the said server through the said central gateway; and
wherein the said clinical and said non-clinical data at the said server is accessed by the said functional stake holders; and
wherein the said clinical and said non-clinical data through the said central gateway is transmitted to the said sponsors.
